(19) United States
(12) Patent Application Publication
(10) Pub. No.: US 2019/0001088 A1
(43) Pub. Date: Jan. 3, 2019

(54) NASAL DELIVERY ASSEMBLY FOR A FLUID PRODUCT AND METHOD FOR ACTUATING THE ASSEMBLY

(71) Applicant: APTAR FRANCE SAS, Le Neubourg (FR)

(72) Inventor: Ludovic PETIT, Vitot (FR)

(73) Assignee: APTAR FRANCE SAS, Le Neubourg (FR)

(21) Appl. No.: 16/064,711

(22) PCT Filed: Jan. 6, 2017
(86) PCT No.: PCT/FR2017/050038

§ 371 Date: Jun. 21, 2018

(30) Foreign Application Priority Data
Jan. 7, 2016 (FR) 1650121

(51) Int. Cl. A61M 15/08; A61M 11/00; A61M 15/00; B05B 11/06; B05B 7/04; B05B 7/12; G01F 11/02

(52) U.S. Cl. CPC A61M 15/08; A61M 11/007; A61M 15/0003; A61M 15/0065; B05B 11/3019; B05B 11/061; B05B 7/0483; B05B 7/1209; G01F 11/028; A61M 15/009

(57) ABSTRACT

A nasal fluid dispenser assembly (100) having a first reservoir (110) of fluid, a metering pump (120) mounted on the first reservoir (110) and including a piston (121); a compressed gas flow generator system; a nasal endpiece (150) having a connection portion (151) fastened to the outlets of the pump and valve (140), a fluid chamber (153) that receives the dose of fluid and the flow of compressed gas, and a nasal insertion element (152 that extends longitudinally along an axis (B) and contains a dispenser channel (154) connected to the fluid chamber and has a dispenser orifice (155). The axis (B) forms an angle (α) relative to the longitudinal axis (A). The dose of fluid is dispensed into the fluid chamber and then expelled under pressure by the flow of compressed gas out from the fluid chamber through the dispenser channel (154) the dispenser orifice (155).

The present invention relates to a nasal fluid dispenser assembly and to a method of actuating such a dispenser assembly.

Nasal fluid dispenser devices are well known. They generally comprise: a reservoir containing one or more doses of fluid in the form of liquid, gel, foam, or fluid; dispenser means such as a pump, a valve, or an air expeller; and a nasal dispenser head for inserting into a user's nostril, said nasal dispenser head including a dispenser orifice. When the dispenser device is actuated, a dose of fluid is dispensed into a user's nostril.

A drawback with those prior-art devices relates to dispensing the dose of fluid into the nostril.

In known manner, starting from the nostril orifice, a nostril comprises, in particular: the nasal valve; the bottom, intermediate, and top conchae; the frontal sinus; and the ethmoid sinuses. The nasal valve has a particular shape. It extends over about 1 centimeter (cm) in depth, has a vertical longitudinal section of about 3 cm to 4 cm, and a width of about 1 millimeter (mm) to 3 mm. Beyond the nasal valve, the nasal cavity includes a larger cavity (about 7 cm in height by 2 cm to 3 cm in width). The conchae face the nasal valve. The roof of the nasal cavity is situated above the conchae, which roof includes the ethmoid sinuses, the olfactory bulb, and the olfactory nerve.

Since nasal dispenser devices are non-invasive, or only minimally invasive, the dispensed fluid generally does not pass through the nasal valve. Thus, as a result of the anatomy of the nasal valve and of the protective location of the conchae, the axial or rectilinear path of the fluid spray particles does not make it possible to reach the roof of the nasal cavity, and in particular the ethmoid sinuses.

Documents U.S. Pat. No. 5,437,267, U.S. Pat. No. 3,921,857, FR 2 257 352, WO 2004/011071, CA 985 232, FR 2 852 928, DE 10 2013 220 492, and WO 02/074372 describe prior-art devices, mainly two-phase pumps, in which a flow of compressed air is mixed with the flow of liquid at the dispenser orifice so as to provide spraying.

An object of the present invention is to provide a nasal fluid dispenser assembly and/or a method of actuation that do not have the above-mentioned drawbacks.

Another object of the present invention is to provide a nasal fluid dispenser assembly and/or a method of actuation that improve(s) the percentage of dose that reaches the ethmoid sinuses.

Another object of the present invention is to provide a nasal fluid dispenser assembly that is simple and inexpensive to manufacture and to assemble.

The present invention thus provides a nasal fluid dispenser assembly comprising:
a first reservoir containing a plurality of doses of a fluid, a metering pump being mounted on said first reservoir so as to dispense a dose of said fluid each time said dispenser assembly is actuated, said metering pump including a piston that is movable axially along a longitudinal axis A;
a compressed gas flow generator system for dispensing a flow of compressed gas each time said dispenser assembly is actuated;
a nasal endpiece comprising a connection portion and a nasal insertion element, said connection portion being fastened at one end to the outlet of said metering pump, and at the other end to the outlet of said compressed gas flow generator system, said connection portion including a fluid chamber that, each time said dispenser assembly is actuated, receives the dose of fluid dispensed by said metering pump, and the flow of compressed gas dispensed by said compressed gas flow generator system, said nasal insertion element comprising an elongate body for inserting into a user's nostril, said elongate body extending longitudinally along an axis B and containing a dispenser channel that is connected at one end to said fluid chamber, and that is provided at the other end with a dispenser orifice through which said dose of fluid and said flow of compressed gas are dispensed, said axis B forming an angle α relative to said longitudinal axis A;

wherein, said dose of fluid is initially dispensed into said fluid chamber, and then said dose of fluid is expelled under pressure by said flow of compressed gas out from said fluid chamber, through said dispenser channel, and through said dispenser orifice.

Advantageously, said metering pump is actuated before said compressed gas flow generator system.

Advantageously, the actuation force of said metering pump is less than the actuation force of said compressed gas flow generator system.

In an advantageous variant, the actuation stroke of said metering pump is shorter than the actuation stroke of said compressed gas flow generator system, such that in the event of simultaneous actuations, said dose of fluid is dispensed by said metering pump before said flow of compressed gas is dispensed by said compressed gas flow generator system.

In another advantageous variant, said compressed gas flow generator system includes locking means that are released after said dose of fluid has been dispensed by said metering pump.

Advantageously, said locking means prevent said compressed gas flow generator system from being actuated.

Advantageously, said locking means prevent said compressed gas flow from being dispensed.

Advantageously, said compressed gas flow generator system includes a second reservoir containing a plurality of doses of at least one pressurized propellant gas, a metering valve being mounted on said second reservoir so as to dispense a dose of propellant gas each time said dispenser assembly is actuated, said metering valve including a valve member that is movable axially along said longitudinal axis A.

Advantageously, said piston of said metering pump co-operates with a pump spring that urges said piston towards a rest position, and said valve member of said metering valve co-operates with a valve spring that urges said valve member towards said rest position, said pump spring resisting deformation less than said valve spring.

Advantageously, during actuation of said pump assembly, said first and second reservoirs are moved axially towards each other, thus urging said piston and said valve member towards respective actuated positions, said piston being moved towards its actuated position before said valve member is moved towards its actuated position.

Advantageously, said second reservoir, in addition to said propellant gas, contains a second fluid that is adapted to be combined in the nostril with said fluid coming from said first reservoir.

Advantageously, said dispenser assembly includes an actuator that is fastened around said second reservoir and/or said compressed gas flow generator system, said actuator including a finger rest for receiving the user's fingers while actuating said dispenser assembly.

Advantageously, said actuator comprises a hollow shell that contains said compressed gas flow generator system and that surrounds said metering pump, at least in part, said first reservoir projects axially out from said hollow shell.

In an advantageous variant, said actuator comprises a hollow sleeve that is fastened around said compressed gas flow generator system and that surrounds said metering pump, at least in part, said first reservoir and said compressed gas flow generator system project axially out from said hollow shell.

Advantageously, said fluid contained in said first reservoir includes an active pharmaceutical.

Advantageously, said nasal insertion element of said nasal endpiece is made, at least in part, of a flexible and/or deformable material, such as a thermoplastic material.

Advantageously, the portion of said nasal insertion element that is inserted into the nostril during actuation is made with said flexible and/or deformable material.

The present invention also provides a method of actuating a nasal fluid dispenser assembly, the method being characterized in that it includes the following steps:

(a) providing a nasal fluid dispenser assembly comprising:
a first reservoir containing a plurality of doses of a fluid, a metering pump being mounted on said first reservoir so as to dispense a dose of said fluid each time said dispenser assembly is actuated, said metering pump including a piston that is movable axially along a longitudinal axis;
a compressed gas flow generator system for dispensing a flow of compressed gas each time said dispenser assembly is actuated;
a nasal endpiece comprising a connection portion and a nasal insertion element, said connection portion being fastened at one end to the outlet of said metering pump, and at the other end to the outlet of said compressed gas flow generator system, said connection portion including a fluid chamber that, each time said dispenser assembly is actuated, receives the dose of fluid dispensed by said metering pump, and the flow of compressed gas dispensed by said compressed gas flow generator system, said nasal insertion element comprising an elongate body for inserting into a user's nostril, said elongate body extending longitudinally along an axis and containing a dispenser channel that is connected at one end to said fluid chamber, and that is provided at the other end with a dispenser orifice through which said dose of fluid and said flow of compressed gas are dispensed, said axis forming an angle α relative to said longitudinal axis;

(b) actuating said metering pump before said compressed gas flow generator system, such that said dose of fluid is initially dispensed into said fluid chamber, and then said dose of fluid is expelled under pressure by said flow of compressed gas out from said fluid chamber, through said dispenser channel, and through said dispenser orifice.

Advantageously, the actuation force of said metering pump is less than the actuation force of said compressed gas flow generator system.

Advantageously, the actuation stroke of said metering pump is shorter than the actuation stroke of said compressed gas flow generator system, such that in the event of simultaneous actuations, said dose of fluid is dispensed by said metering pump before said flow of compressed gas is dispensed by said compressed gas flow generator system.

Advantageously, said compressed gas flow generator system includes a second reservoir containing a plurality of doses of at least one pressurized propellant gas, a metering valve being mounted on said second reservoir so as to dispense a dose of propellant gas each time said dispenser assembly is actuated, said metering valve including a valve member that is movable axially along said longitudinal axis, such that during actuation of said dispenser assembly, said first and second reservoirs are moved axially towards each other, thus urging said piston and said valve member towards respective actuated positions, said piston being moved towards its actuated position before said valve member is moved towards its actuated position.

These characteristics and advantages and others appear more clearly from the following detailed description, given by way of non-limiting examples, and with reference to the accompanying drawings, and in which:

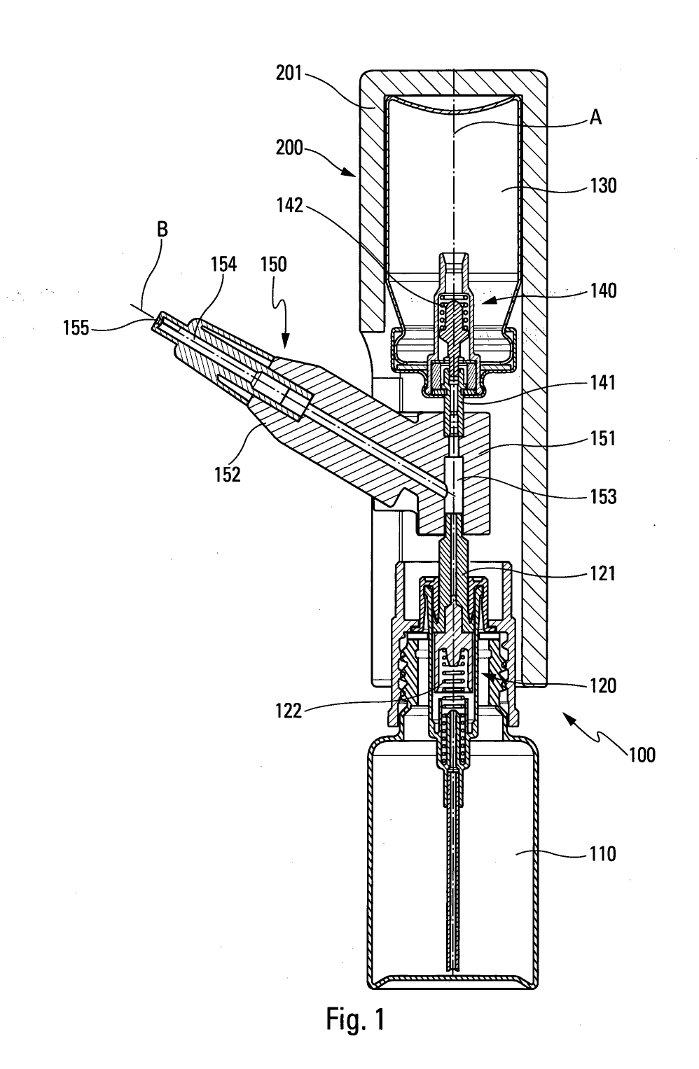

FIG. 1 is a diagrammatic section view of an advantageous embodiment of a nasal fluid dispenser assembly in its rest position;

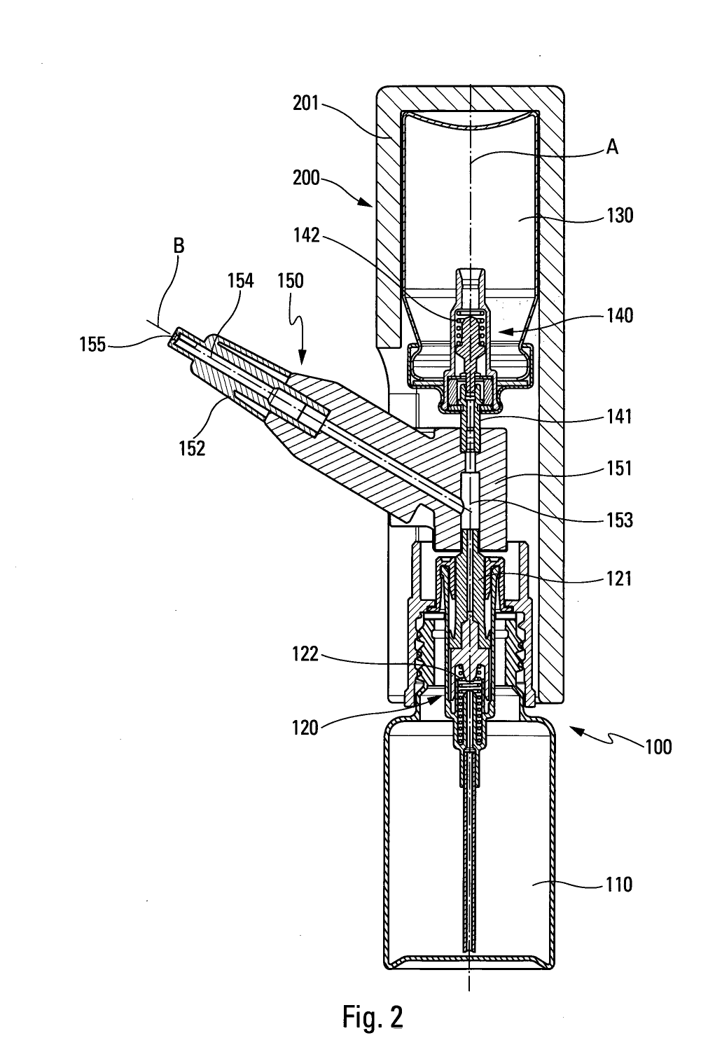

FIG. 2 is a diagrammatic view of the FIG. 1 dispenser assembly, after actuation of the pump and before actuation of the valve;

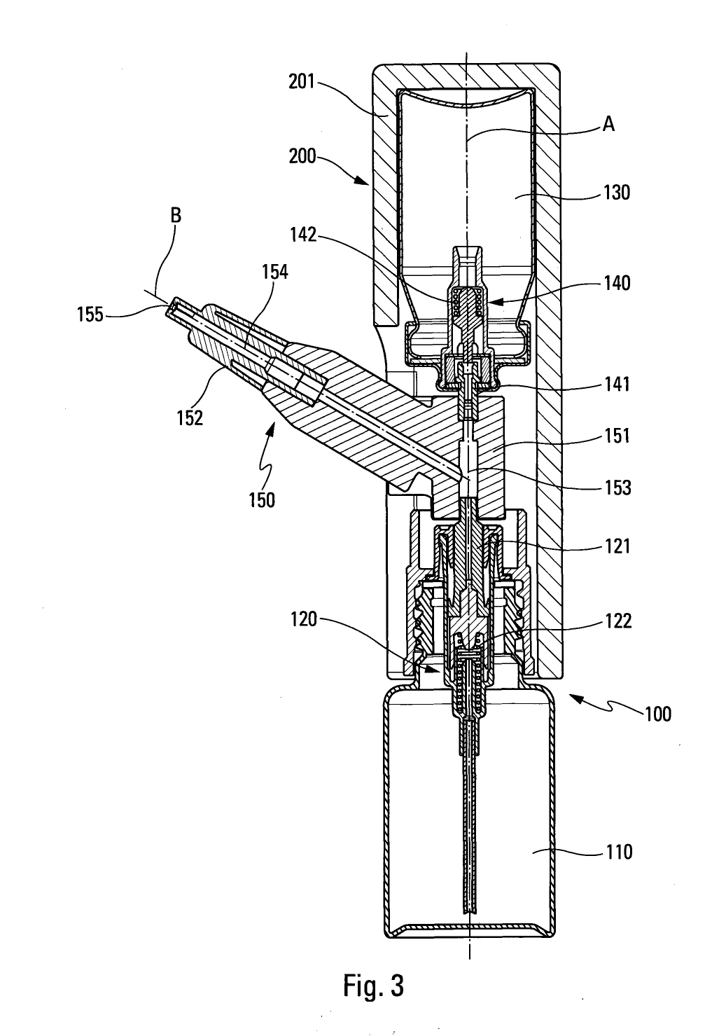

FIG. 3 is a diagrammatic view of the dispenser assembly in FIGS. 1 and 2, after actuation of the valve;

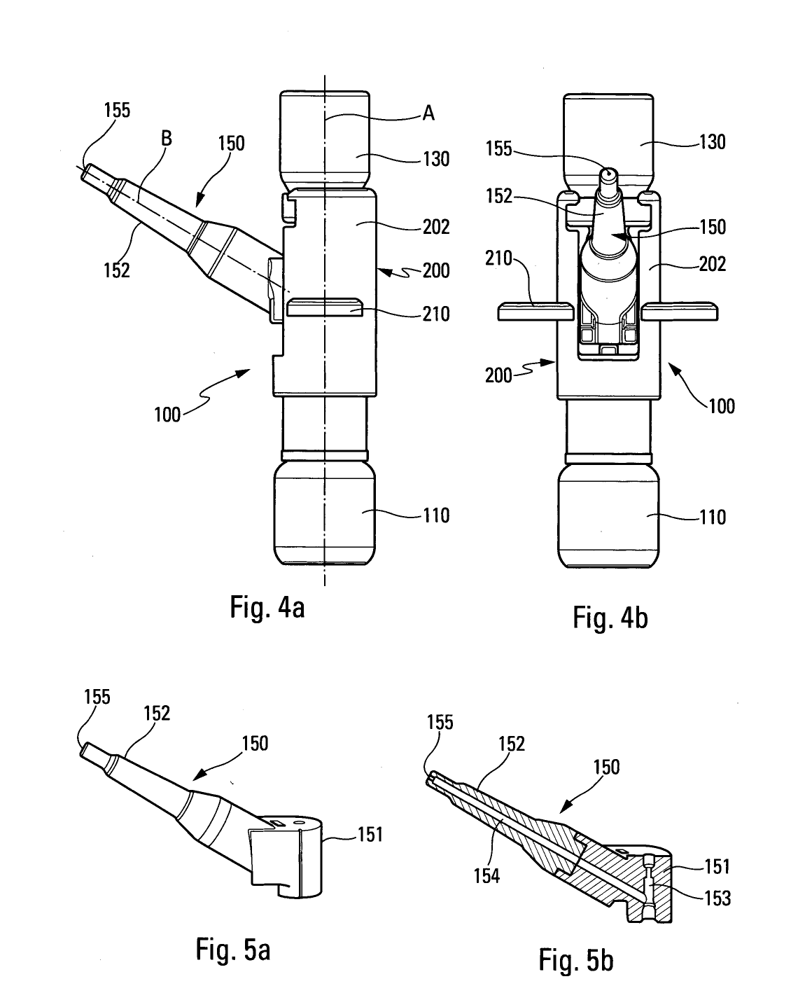

FIGS. 4a and 4b are diagrammatic views, respectively from the side and from the front, of another advantageous embodiment of a dispenser assembly in its rest position;

FIGS. 5a and 5b are diagrammatic side views, respectively in perspective and in section, of an advantageous variant embodiment of the nasal endpiece of the dispenser assemblies in FIGS. 1 to 4;

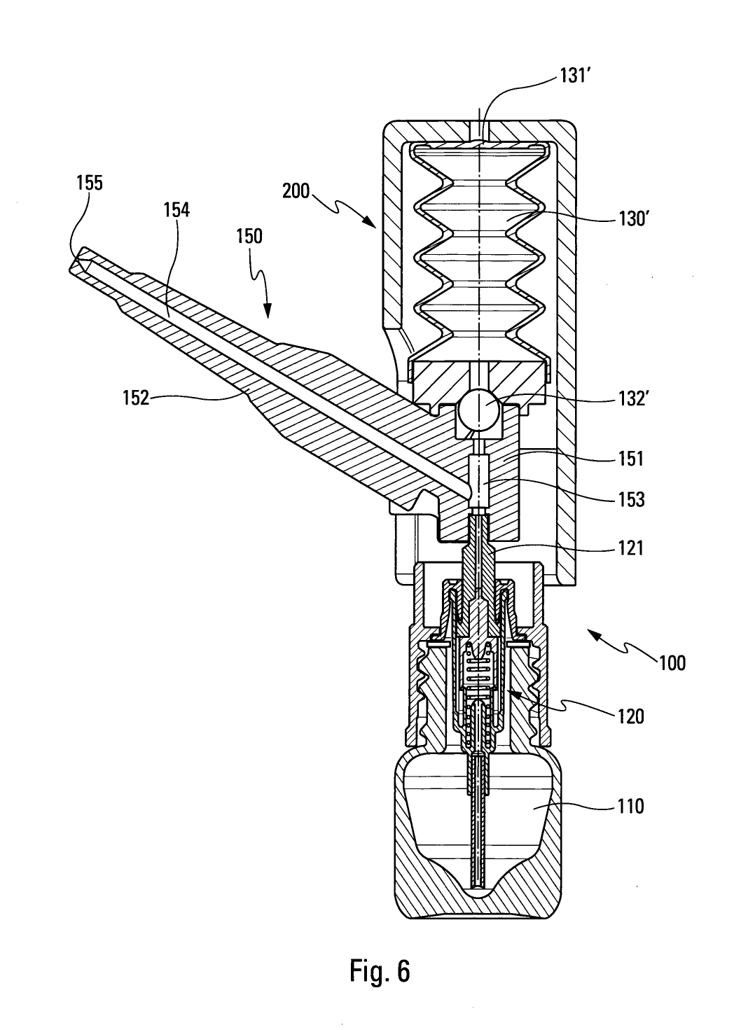

FIG. 6 is a diagrammatic section view of another advantageous embodiment of a nasal fluid dispenser assembly in its rest position;

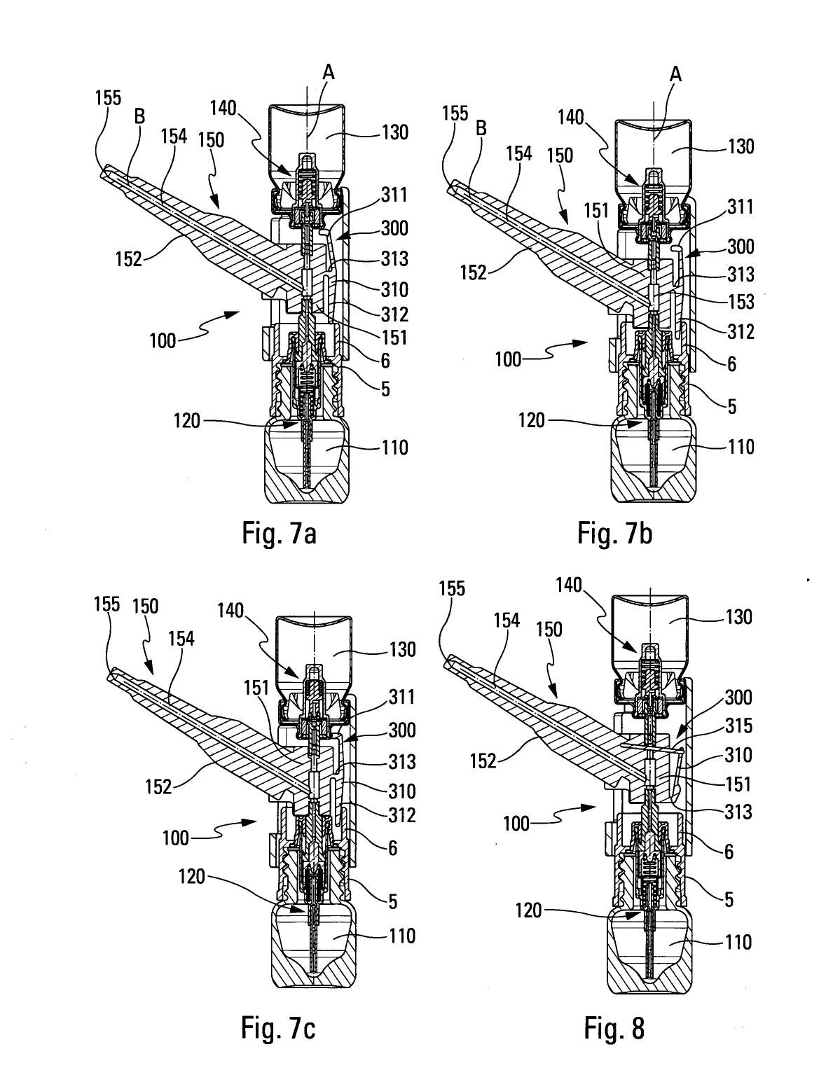

FIGS. 7a to 7c are diagrammatic views similar to the views in FIGS. 1 to 3, showing still another advantageous embodiment;

FIG. 8 is a view similar to the view in FIG. 7c, showing a variant embodiment; and

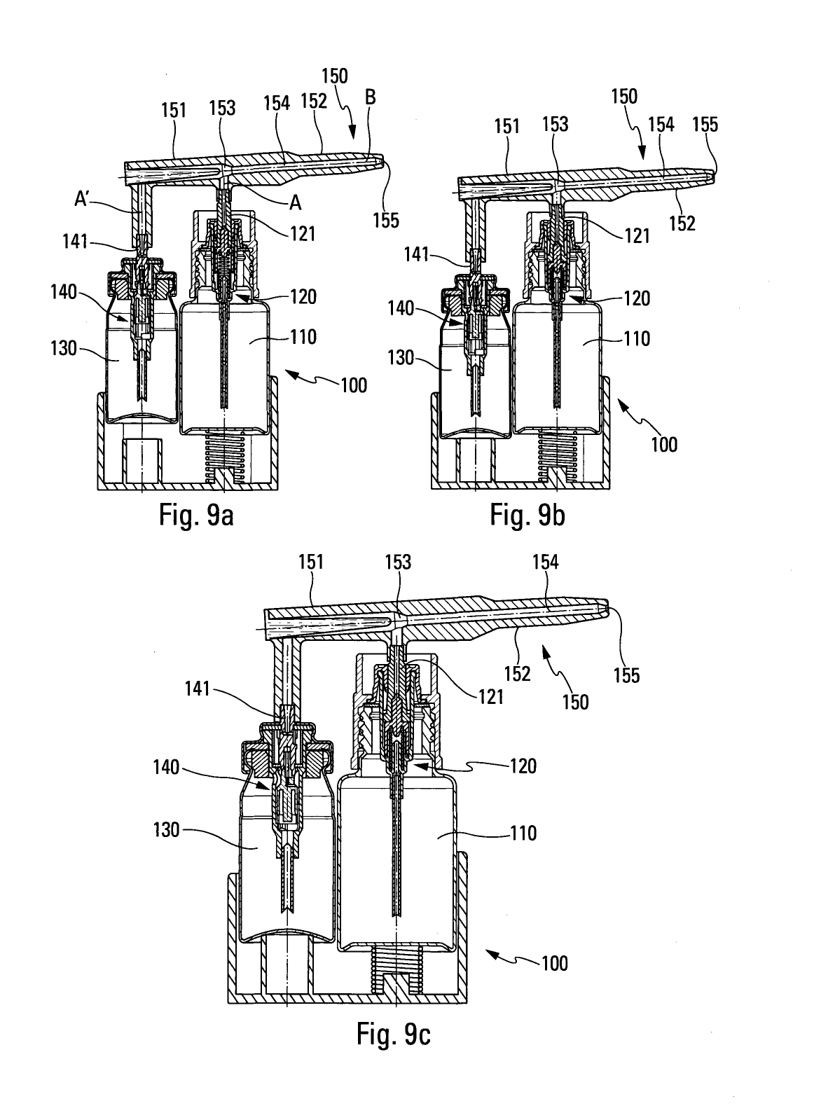

FIGS. 9a to 9c are diagrammatic views similar to the views in FIGS. 1 to 3, showing still another advantageous embodiment.

In the description, the terms “axial” and “radial” are relative either to the longitudinal axis A of the dispenser assembly, or to the longitudinal axis B of the nasal endpiece, the longitudinal axes being shown in FIGS. 1 to 4a. The terms “proximal” and “distal” are relative to the dispenser orifice of said nasal endpiece.

FIGS. 1 to 3 show a first advantageous embodiment.

The nasal fluid dispenser assembly 100 includes a first reservoir 110 containing a plurality of doses of a fluid, typically a fluid including an active pharmaceutical.

A metering pump 120 is mounted on said first reservoir 110 so as to dispense a dose of said fluid each time said dispenser assembly 100 is actuated. In known manner, said metering pump 120 includes a piston 121 that, during actuation of said dispenser assembly 100, moves axially along a longitudinal axis A of said dispenser assembly 100. The metering pump 120 may be mounted on said first reservoir 110 by means of a fastener ring, e.g. screw-fastenable or snap-fastenable on the neck of said first reservoir. However, the structure of the metering pump 120 and its fastening to said first reservoir 110 may be implemented in any known manner, and the present invention is not limited to the embodiments shown in the drawings.

The dispenser assembly 100 also includes a compressed gas flow generator system that is adapted to dispense a flow of compressed gas on each actuation.

In a preferred embodiment, shown in FIGS. 1 to 3 and 7a to 9c, the compressed gas flow generator system includes a second reservoir 130 containing a plurality of doses of at least one pressurized and/or liquefied propellant gas. Advantageously, said second reservoir 130 contains propellant gas only. However, in a variant, in addition to said propellant gas, said second reservoir 130 may contain a second fluid that is adapted to be combined in the nostril with said fluid coming from said first reservoir 110.

A metering valve 140 is mounted on said second reservoir 130 so as to dispense a dose of propellant gas each time said dispenser assembly 100 is actuated. In known manner, said metering valve 140 includes a valve member 141 that, during actuation of said dispenser assembly 100, moves axially along said longitudinal axis A. The metering valve 140 may be mounted on said second reservoir 130 by means of a fastener cap, e.g. crimpable on the neck of said second reservoir. However, the structure of the metering valve 140 and its fastening to said second reservoir 130 may be implemented in any known manner, and the present invention is not limited to the embodiments shown in the drawings.

In a variant to using a metering valve mounted on a reservoir containing propellant gas, it is possible to envisage an air expeller that, on each actuation, compresses the air in a chamber and delivers a flow of compressed air. FIG. 6 shows an example of an air expeller of this type, with an air chamber 130′, advantageously formed by a bellows. The air chamber 130′ preferably includes an inlet valve 131′, in this embodiment formed by a membrane that allows air to be taken in after each actuation, and an outlet valve 132′, in this embodiment formed by a ball. Other embodiments are however possible.

The dispenser assembly 100 further includes a nasal endpiece 150 that comprises a connection portion 151 and a nasal insertion element 152.

Said connection portion 151 is fastened at one end to the outlet of said metering pump 120, and at the other end to the outlet of said metering valve 140. It includes a fluid chamber 153 that, each time said dispenser assembly 100 is actuated, receives both the dose of fluid dispensed by said metering pump 120, and also the flow of compressed gas dispensed by said compressed gas flow generator system.

Said nasal insertion element 152 comprises an elongate body for inserting into a user's nostril. Said elongate body extends longitudinally along an axis B and contains a dispenser channel 154 that is connected at one end to said fluid chamber 153, and that is provided at the other end with a dispenser orifice 155 through which said dose of fluid and said flow of compressed gas are dispensed. As can be seen in the figures, said axis B forms an angle α relative to said longitudinal axis A. Advantageously, said angle α is greater than 30° and less than 90°. In the embodiments in FIGS. 1 to 8, the angle is preferably about 45°.

In the invention, said dose of fluid is initially dispensed into said fluid chamber 153, and then said dose of fluid is expelled under pressure by said flow of compressed gas out from said fluid chamber 153, through said dispenser channel 154, and through said dispenser orifice 155.

Advantageously, the actuation force of said metering pump 120 is less than the actuation force of said compressed gas flow generator system. Thus, during actuation of said dispenser assembly 100, said metering pump 120 is actuated before said compressed gas flow generator system.

Advantageously, the dimensions of the fluid chamber 153, of the outlet of the metering pump 120, and of the dispenser channel 154 are such that when the flow of compressed gas arrives under pressure in said fluid chamber 153, all (or almost all) of the flow of compressed gas flows through said dispenser channel 154, entraining all (or almost all) of said dose of fluid therewith, and this empties and purges not only said fluid chamber 153 but also said dispenser channel 154. In particular, the small radial dimension of the pump outlet prevents compressed gas and/or fluid from being reinjected into said metering pump 120, the flow of compressed gas seeking directly to flow along the path of least resistance, namely said dispenser channel 154 of the nasal endpiece 150.

In known manner, said piston 121 of the pump 120 co-operates with a pump spring 122 that urges said piston 121 towards its rest position. In addition, said valve member 141 of the valve 140 co-operates with a valve spring 142 that urges said valve member 141 towards its rest position. Advantageously, the resistance of said pump spring 122 to deforming is less than the resistance of said valve spring 142 to deforming, which ensures that the pump 120 is actuated first, then the valve 140. Naturally, parameters other than the springs of the pump 120 and of the valve 140 may influence the actuation force.

In the embodiment shown in the figures, in order to actuate said pump assembly 100, said first and second reservoirs 110, 130 are moved axially towards each other, thus urging said piston 121 and said valve member 141 towards their respective actuated positions. It is said piston 121 that moves initially towards its actuated position, before said valve member 141 also moves towards its actuated position.

In a variant, in order to ensure that the fluid is dispensed first, followed by the flow of compressed gas, provision may also be made for the actuation stroke of the metering pump 120 to be shorter than the actuation stroke of the compressed gas flow generator system. Thus, in the event of simultaneous actuations, said dose of fluid is dispensed by said metering pump 120 before said flow of compressed gas is dispensed by said compressed gas flow generator system.

In another variant, shown in FIGS. 7a to 7c and 8, said compressed gas flow generator system may include locking means 300 that are released after said dose of fluid has been dispensed by said metering pump 120. The locking means may prevent said compressed gas flow generator system from being actuated and/or prevent said compressed gas flow from being dispensed.

In the embodiment in FIGS. 7a to 7c, the locking means are formed by a rod 310 that is pivotally mounted via a hinge 313 on the connection portion 151. The rod 310 includes an upper branch 311 that co-operates with the second reservoir 130 and/or the valve 140 so as to prevent said compressed gas flow generator system from being actuated. The rod 310 also includes a lower branch 312 that co-operates with an axial extension 6 of the fastener ring 5 that fastens the pump 12 on the first reservoir 110. At the end of the actuation stroke of said pump 120, said extension 6 co-operates with the lower branch 312 so as to cause it to pivot about the hinge 313, and this automatically causes the upper branch 311 to pivot away from its blocking position, thereby enabling said compressed gas flow generator system to be actuated.

In the embodiment in FIG. 8, it is not the actuation of said compressed gas flow generator system that is blocked, but the dispensing of said flow of compressed gas that is blocked. In this embodiment, the locking means 300 comprise a slide valve 315 that is secured to a rod 310 that is pivotally mounted via a hinge 313 on said connection portion 151. At rest, said slide valve 315 obstructs the outlet of the valve 140. At the end of the actuation stroke of said pump 120, said extension 6 of the fastener ring 5 of the pump 120 co-operates with the rod 310 so as to cause it to pivot about the hinge 313, and this automatically causes the upper branch 311 to pivot away from its blocking position, thereby causing said slide valve to slide out from its blocking position, thereby enabling said flow of compressed gas to flow into said fluid chamber.

In order to facilitate actuation, an actuator 200 may advantageously be provided. The actuator 200 includes a finger rest 210 for receiving the user's fingers while actuating said dispenser assembly, and is fastened around said second reservoir 130 and/or said metering valve 140. Such an actuator could also be secured to an air expeller that is adapted to generate a flow of compressed air on each actuation.

During actuation, the user places one or two fingers on said finger rest 210 and the thumb below said first reservoir 110, and urges the two elements towards each other. This initially moves the piston 121 and thus actuates the metering pump 120, then, when the dose of fluid has been transferred into said fluid chamber 153, continuing the actuation force actuates the compressed gas flow generator system, and this causes a flow of compressed gas to be expelled, which in turn expels the dose of fluid through the dispenser orifice 155.

In the embodiment in FIGS. 1 to 3, said actuator 200 comprises a hollow shell 201 that contains said second reservoir 130 and that surrounds said metering pump 120, at least in part, such that said first reservoir 110 projects axially out from said hollow shell 201. In this variant, the second reservoir 130 is not visible from the outside.

In the embodiment in FIGS. 4a and 4b, said actuator 200 comprises a hollow sleeve 202 that is fastened around said metering valve 140, e.g. at the fastener cap, surrounding said metering pump 120, at least in part. In this embodiment, the first and second reservoirs 110, 130 project axially out from said hollow sleeve 202. This embodiment makes it possible to reduce the external dimensions of said dispenser assembly, and to access the second reservoir 130.

Advantageously, as a result of using compressed gas, said dispenser orifice 155 may be a mere opening at the axial end of the dispenser channel 154, without it being necessary to provide a spray profile for generating a spray.

In the embodiment in FIGS. 1 to 3, the nasal endpiece 150 is made in rigid manner. FIGS. 5a and 5b show an advantageous variant, in which the nasal insertion element 152 of said nasal endpiece 150 is made, at least in part, of a flexible and/or deformable material, such as a thermoplastic material. Advantageously, all of said nasal insertion element 152 that is inserted into the nostril during actuation is made of the flexible and/or deformable material. This makes it possible not only to improve the comfort of the user, but also to improve the effectiveness of said assembly, since a flexible nasal endpiece can be inserted further into the nostril, and may self-orientate in the nasal valve, even when said nasal endpiece is not at its best insertion angle.

FIGS. 9a to 9c show still another advantageous embodiment, in which the first and second reservoirs 110, 130 are arranged one next to the other. In this embodiment, the pump 120 is thus actuated along a longitudinal axis A, and the valve 140 is actuated along a longitudinal axis A′ that is parallel to said axis A. As with the other embodiments, the invention envisages initially actuating the pump 120 for initially dispensing the dose of fluid into the fluid chamber 153, and then actuating the valve 140 so as to expel said dose into the nostril.

The present invention is described above with reference to advantageous embodiments, but naturally any modification could be applied thereto by a person skilled in the art, without going beyond the ambit of the present invention, as defined by the accompanying claims.

1. A nasal fluid dispenser assembly, characterized in that it comprises:
a first reservoir containing a plurality of doses of a fluid, a metering pump being mounted on said first reservoir so as to dispense a dose of said fluid each time said dispenser assembly is actuated, said metering pump including a piston that is movable axially along a longitudinal axis (A);
a compressed gas flow generator system for dispensing a flow of compressed gas each time said dispenser assembly is actuated;
a nasal endpiece comprising a connection portion and a nasal insertion element, said connection portion being fastened at one end to the outlet of said metering pump, and at the other end to the outlet of said compressed gas flow generator system, said connection portion including a fluid chamber that, each time said dispenser assembly is actuated, receives the dose of fluid dispensed by said metering pump, and the flow of compressed gas dispensed by said compressed gas flow generator system, said nasal insertion element comprising an elongate body for inserting into a user's nostril, said elongate body extending longitudinally along an axis and containing a dispenser channel that is connected at one end to said fluid chamber, and that is provided at the other end with a dispenser orifice through which said dose of fluid and said flow of compressed gas are dispensed, said axis (B) forming an angle (α) relative to said longitudinal axis (A);
wherein, during actuation of said dispenser assembly, said metering pump is actuated before said compressed gas flow generator system, such that said dose of fluid is initially dispensed into said fluid chamber, and then said dose of fluid is expelled under pressure by said flow of compressed gas out from said fluid chamber, through said dispenser channel, and through said dispenser orifice.

2. A dispenser assembly according to claim 1, wherein the actuation force of said metering pump is less than the actuation force of said compressed gas flow generator system.

3. A dispenser assembly according to claim 1, wherein the actuation stroke of said metering pump his shorter than the actuation stroke of said compressed gas flow generator system, such that in the event of simultaneous actuations, said dose of fluid is dispensed by said metering pump before said flow of compressed gas is dispensed by said compressed gas flow generator system.

4. A dispenser assembly according to claim 1, wherein said compressed gas flow generator system includes locking means that are released after said dose of fluid has been dispensed by said metering pump.

5. A dispenser assembly according to claim 4, wherein said locking means prevent said compressed gas flow generator system from being actuated.

6. A dispenser assembly according to claim 4, wherein said locking means prevent said compressed gas flow from being dispensed.

7. A dispenser assembly according to claim 1, wherein said compressed gas flow generator system includes a second reservoir containing a plurality of doses of at least one pressurized propellant gas, a metering valve being mounted on said second reservoir so as to dispense a dose of propellant gas each time said dispenser assembly is actuated, said metering valve including a valve member that is movable axially along said longitudinal axis (A).

8. A dispenser assembly according to claim 7, wherein said piston of said metering pump co operates with a pump spring that urges said piston towards a rest position, and said valve member of said metering valve co-operates with a valve spring that urges said valve member towards said rest position, said pump spring resisting deformation less than said valve spring.

9. A dispenser assembly according to claim 7, wherein, during actuation of said dispenser assembly, said first and second reservoirs are moved axially towards each other, thus urging said piston and said valve member towards respective actuated positions, said piston being moved towards its actuated position before said valve member is moved towards its actuated position.

10. A dispenser assembly according to claim 7, wherein said second reservoir, in addition to said propellant gas, contains a second fluid that is adapted to be combined in the nostril with said fluid coming from said first reservoir.

11. A dispenser assembly according to claim 8, including an actuator that is fastened around said second reservoir and/or said compressed gas flow generator system, said actuator including a finger rest for receiving the user's fingers while actuating said dispenser assembly.

12. A dispenser assembly according to claim 11, wherein said actuator comprises a hollow shell that contains said compressed gas flow generator system and that surrounds said metering pump, at least in part, said first reservoir projects axially out from said hollow shell.

13. A dispenser assembly according to claim 11, wherein said actuator comprises a hollow sleeve that is fastened around said compressed gas flow generator system and that surrounds said metering pump at least in part, said first reservoir and said compressed gas flow generator system project axially out from said hollow shell.

14. A dispenser assembly according to claim 1, wherein said fluid contained in said first reservoir includes an active pharmaceutical.

15. A dispenser assembly according to claim 1, wherein said nasal insertion element of said nasal endpiece is made, at least in part, of a flexible and/or deformable material, such as a thermoplastic material.

16. A dispenser assembly according to claim 15, wherein the portion of said nasal insertion element that is inserted into the nostril during actuation is made with said flexible and/or deformable material.

17. A method of actuating a nasal fluid dispenser assembly, the method being characterized in that it includes the following steps:
(a) providing a nasal fluid dispenser assembly comprising:
a first reservoir containing a plurality of doses of a fluid, a metering pump being mounted on said first reservoir so as to dispense a dose of said fluid each time said dispenser assembly is actuated, said metering pump including a piston that is movable axially along a longitudinal axis;
a compressed gas flow generator system for dispensing a flow of compressed gas each time said dispenser assembly is actuated;
a nasal endpiece comprising a connection portion and a nasal insertion element, said connection portion being fastened at one end to the outlet of said metering pump, and at the other end to the outlet of said compressed gas flow generator system, said connection portion including a fluid chamber that, each time said dispenser assembly is actuated, receives the dose of fluid dispensed by said metering pump, and the flow of compressed gas dispensed by said compressed gas flow generator system, said nasal insertion element comprising an elongate body for inserting into a user's nostril, said elongate body extending longitudinally along an axis (B) and containing a dispenser channel that is connected at one end to said fluid chamber, and that is provided at the other end with a dispenser orifice through which said dose of fluid and said flow of compressed gas are dispensed, said axis (B) forming an angle (α) relative to said longitudinal axis (A);
(b) actuating said metering pump before said compressed gas flow generator system, such that said dose of fluid is initially dispensed into said fluid chamber, and then said dose of fluid is expelled under pressure by said flow of compressed gas out from said fluid chamber, through said dispenser channel, and through said dispenser orifice.

18. A method according to claim 17, wherein the actuation force of said metering pump is less than the actuation force of said compressed gas flow generator system.

19. A method according to claim 17, wherein the actuation stroke of said metering pump is shorter than the actuation stroke of said compressed gas flow generator system, such that in the event of simultaneous actuations, said dose of fluid is dispensed by said metering pump before said flow of compressed gas is dispensed by said compressed gas flow generator system.

20. A method according to claim 17, wherein said compressed gas flow generator system includes a second reservoir containing a plurality of doses of at least one pressurized propellant gas, a metering valve being mounted on said second reservoir so as to dispense a dose of propellant gas each time said dispenser assembly is actuated, said metering valve including a valve member that is movable axially along said longitudinal axis (A), such that during actuation of said dispenser assembly, said first and second reservoirs are moved axially towards each other, thus urging said piston and said valve member towards respective actuated positions, said piston being moved towards its actuated position before said valve member is moved towards its actuated position.
